(19) United States
(12) Patent Application Publication
(10) Pub. No.: US 2012/0247964 A1
(43) Pub. Date: Oct. 4, 2012

(54) MICROFLUIDIC CONTROL DEVICE FOR MEASURING GLYCOSLYATED HEMOGLOBIN, METHOD FOR MANUFACTURING THE SAME AND METHOD FOR OPERATING THE SAME

(75) Inventors: Dae Sik LEE, Daejeon (KR); Moon Youn Jung, Daejeon (KR); Seung Hwan Kim, Daejeon (KR)

(73) Assignee: Electronics and Telecommunications Research Institute, Daejeon (KR)

(21) Appl. No.: 13/436,753

(22) Filed: Mar. 30, 2012

(30) Foreign Application Priority Data
Apr. 1, 2011 (KR) 10-2011-0030232
Sep. 15, 2011 (KR) 10-2011-0092825

(51) Int. Cl. G01N 27/327; G01N 33/66; B81B 1/00; C25D 1/10

(52) U.S. Cl. 205/70; 204/403.14; 205/777.5

(57) ABSTRACT

Disclosed is a microfluidic control device which may electrochemically and simply measure the value of glycosylated hemoglobin in blood on a chip, a method for manufacturing the same, and a method for operating the same.

### CROSS-REFERENCE TO RELATED APPLICATIONS

This application is based on and claims priority from Korean Patent Application Nos. 10-2011-0030232, filed on Apr. 1, 2011 and 10-2011-0092825, filed on Sep. 15, 2011, with the Korean Intellectual Property Office, the present disclosures of which are incorporated herein in their entireties by reference.

### TECHNICAL FIELD

The present invention relates to a microfluidic control device which may electrochemically and simply measure the value of glycosylated hemoglobin in blood on a chip, a method for manufacturing the same, and a method for operating the same.

### BACKGROUND

Glycosylated hemoglobin (HbA1c) is hemoglobin present in red blood cells and refers to hemoglobin that has bound with glucose. Glycosylated hemoglobin is a type of hemoglobin used to find the long-term concentration of glucose (blood sugar) in blood, and glycosylated hemoglobin of the red blood cells once bound with sugar survives for about 120 days, and thus an average blood sugar concentration over the past 2 to 3 months may be estimated through glycosylated hemoglobin. Accordingly, the value of glycosylated hemoglobin is used as an important index in the observation of treatment process of diabetes and the prediction of future prognosis.

U.S. Pat. No. 6,300,142 discloses an apparatus for optically analyzing glycated protein and other analytes in a bio-sample such as blood, the apparatus allowing the test sample to react with a first reactant through a first injection port and subsequently to react with a second reactant through a second injection port to measure a material to be analyzed. However, according to the Patent Document, it is disadvantageous in that it is difficult to control fluids accurately and rapidly because the fluid transfer is carried out by manual operation and motors and it is difficult to control fluids precisely because mechanical processing is used in the manufacture of the apparatus.

Korean Patent No. 10-0798471 discloses a cassette for measuring glycosylated hemoglobin, including a first receiving zone for receiving a first reagent, a second receiving area for receiving a second reagent, a reaction zone in which a blood sample reacts with the first reagent or the second reagent, and a measuring zone for optically measuring the amount of the total hemoglobin or glycosylated hemoglobin in a blood sample. The cassette for measuring glycosylated hemoglobin according to the Patent Document uses a separate motor driving unit for rotation of the cassette, and thus the structure thereof is complicated, the volume thereof is large, and the reagents are consumed in large amounts. While the first and second reagents are introduced into the first and second receiving zones, respectively, some of the two reagents are mixed. The cassette is manufactured by a process of attaching an upper substrate to a lower substrate in which an internal structure is disposed, and thus reagents may migrate along a minute crack on a binding site of the upper substrate and the lower substrate, thereby generating errors in the measurement result.

Thus, there is need for developing an apparatus for measuring glycosylated hemoglobin, which may control the flow of fluid reproducibly and precisely without any separate power source or manual operation.

### SUMMARY

Therefore, the present invention has been made in view of the above-mentioned problems. It is an object of the present invention to provide a microfluidic control device which may electrochemically and simply measure the value of glycosylated hemoglobin in blood on a chip, a method for manufacturing the same, and a method for operating the same.

According to an aspect of the present invention, there is provided a microfluidic control device, including a first substrate; and a second substrate provided on the first substrate and having a first injection port into which a blood sample is injected, a second injection port into which glucose enzyme is injected, a glucosic acid filter unit which is connected to the first injection port and the second injection port and captures glucosic acid which has been produced by reaction of the blood sample with the glucose enzyme, a sensing chamber unit which is connected to the glucosic acid filter unit and electrochemically measures the concentration of hemoglobin and glycosylated hemoglobin in the blood sample introduced from the glucosic acid filter unit, an exhaust port which is connected to the sensing chamber unit and discharges the blood sample which has completed the reaction in the sensing chamber unit, a valve unit for controlling the flow of fluid between the sensing chamber unit and the exhaust port, and a pump unit for forcingly flowing the blood sample which has completed the reaction in the sensing chamber unit to the exhaust port.

According to another aspect of the present invention, there is provided a method for manufacturing the above-described microfluidic control device for measuring glycosylated hemoglobin, including: preparing a first substrate; preparing a second substrate, including applying a photosensitive photoresist on a template substrate, forming a channel structure corresponding to a glucosic acid filter unit, a sensing chamber unit, a valve unit, and a pump unit on the template substrate by a photolithography process, performing electroplating on the channel structure to form a metal mold and removing the template substrate, transcribing the metal mold on a second precursor substrate, and forming a first injection port, a second injection port, and an exhaust port on the second precursor substrate; and combining the second substrate on the first substrate to form the glucosic acid filter unit, the sensing chamber unit, the valve unit, and the pump unit.

According to still another aspect of the present invention, there is provided a method for operating a microfluidic control device for measuring glycosylated hemoglobin, including injecting a blood sample into a first injection port and injecting glucose enzyme into a second injection port to naturally flow the blood sample and the glucose enzyme to the glucosic acid filter unit by capillary force, by using the above-described microfluidic control device for measuring glycosylated hemoglobin; capturing the glucosic acid produced while passing through the glucosic acid filter unit; transferring the blood sample which has passed through the glucosic acid filter unit to the sensing chamber unit by capillary force and electrochemically measuring concentration of the hemoglobin and the glycosylated hemoglobin; and forcingly flowing the blood sample which has been reacted in the sensing chamber unit to flow to the exhaust port by operating the pump unit.

In according to the embodiments of the present invention, when a microfluidic channel substrate is combined with a sensing substrate, it is possible to makes natural flow by capillary force of a flow channel, forced stop and flow by a micro-valve and a pump, and thus, the flow of the fluid may be reproducibly and precisely controlled without any separate power source or manual operation, thereby leading to an effective quantitative analysis of glycosylated hemoglobin in a trace amount of blood. Also, when glucosic acid which may be an obstacle to the sensing of glycosylated hemoglobin by a glucosic acid filter unit is removed in advance, reliability may be imparted to the analysis. When an electrochemical sensing method is adopted, miniaturization of devices may be implemented, and when a semiconductor process is used, a precise manufacture of the device may be realized, and it is advantageous in mass production.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a perspective view schematically illustrating a microfluidic control device for measuring glycosylated hemoglobin according to an embodiment of the present disclosure.

FIG. 2 is a perspective view schematically illustrating separately an upper substrate and a lower substrate of the microfluidic control device for measuring glycosylated hemoglobin of FIG. 1.

FIGS. 3A to 3C are schematic views schematically illustrating a method for manufacturing a microfluidic control device according to an embodiment of the present invention.

### BRIEF DESCRIPTION OF THE INDICATIONS

| | |
|---|---|
| 100: an upper substrate, | 200: a lower substrate, |
| 101: a first substrate, | 102: a second injection port, |
| 103: a glucosic acid filter unit, | 104: a sensing chamber unit, |
| 105: an exhaust port, | 106: a waste liquid chamber, |
| 107: a valve unit, | 108: a pump unit, |
| 108a: a washing solution storing unit, | |
| 108b: a gas generating unit, | |
| 109: a filter, | 201: sensing electrodes |
| 202: a heater | |

### DETAILED DESCRIPTION

In the following detailed description, reference is made to the accompanying drawing, which form a part hereof The illustrative embodiments described in the detailed description, drawing, and claims are not meant to be limiting. Other embodiments may be utilized, and other changes may be made, without departing from the spirit or scope of the subject matter presented here.

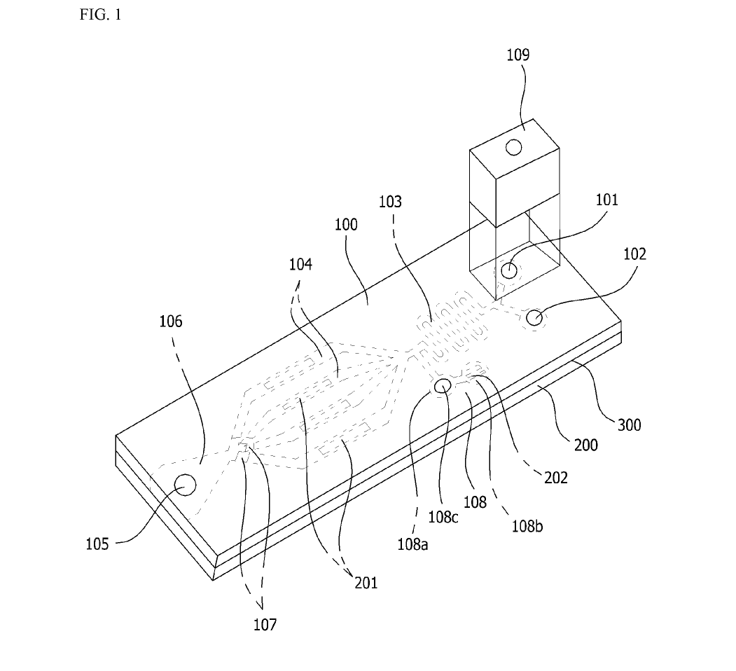

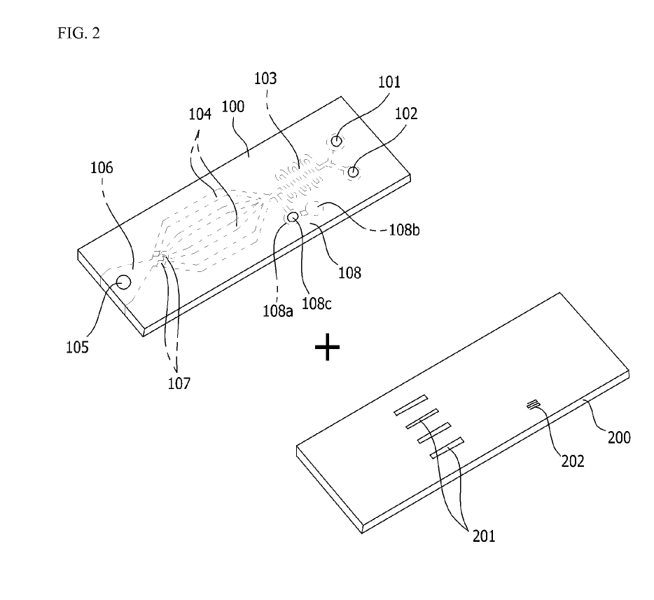

FIG. 1 is a perspective view schematically illustrating a microfluidic control device for measuring glycosylated hemoglobin according to an embodiment of the presentinvention, and FIG. 2 is a perspective view schematically illustrating separately an upper substrate and a lower substrate of the microfluidic control device for measuring glycosylated hemoglobin of FIG. 1. Parts being shown in dotted lines in FIGS. 1 and 2 represent elements which are not exposed to the outside.

Referring to FIGS. 1 and 2, the microfluidic control device for measuring glycosylated hemoglobin according to an embodiment of the present invention includes an upper substrate 100 and a lower substrate 200.

The upper substrate 100 includes a first injection port 101, a second injection port 102, a glucosic acid filter unit 103, a sensing chamber unit 104, an exhaust port 105, a valve unit 107, and a pump unit 108, and the lower substrate 200 includes sensing electrodes 201 and a heater 202.

Each of the upper substrate 100 and the lower substrate 200 may include a polymer substrate formed of at least one polymer material selected from cyclo olefin copolymer (COC), polymethylmethacrylate (PMMA), polycarbonate (PC), cyclo olefin polymer (COP), liquid crystalline polymers (LCP), polydimethylsiloxane (PDMS), polyamide (PA), polyethylene (PE), polyimide (PI), polypropylene (PP), polyphenylene ether (PPE), polystyrene (PS), polyoxymethylene (POM), polyetheretherketone (PEEK), polyether sulfone (PES), polyethylene terephthalate (PET), polytetrafluoroethylene (PTFE), polyvinylchloride (PVC), polyvinylidene fluoride (PVDF), polybutyleneterephthalate (PBT), fluorinated ethylenepropylene (FEP), and perfluoralkoxyalkane (PFA), a glass substrate, a silicon substrate, and the like.

The upper substrate 100 and the lower substrate 200 may be formed of materials having different hydrophobicity and/or hydrophilicity. The upper substrate 100 and the lower substrate 200 may be further provided with films having partially different hydrophobicity and/or hydrophilicity on the surfaces facing each other. This allows the moving speed of a fluid sample to be controlled through micro-molding operation of a flow channel or surface modification of the flow channel.

Each of the upper substrate 100 and the lower substrate 200 may be molded by one type selected from a traditional mechanical processing method such as injection molding, hot embossing, casting, stereolithography, laser ablation, rapid prototyping, silk screen, numerical control machining (NC machining), and the like, or a semiconductor processing method using photolithography, and it is preferred that the photolithography process is used.

A blood sample containing red blood cells derived from whole blood is injected into the first injection port 101 and glucose enzyme is injected into the second injection port 102. The blood sample may additionally include a filter 109 for capturing red blood cells and for separating giant proteins before being injected into the first injection port 101.

The blood sample injected through the first injection port 101 and the glucose enzyme injected through the second injection port 102 naturally flow into the sensing chamber unit 104 by capillary force.

The glucosic acid filter unit 103 is a space unit in the form of channel, in which the blood sample injected through the first injection port 101 and the glucose enzyme injected through the second injection port 102 are mixed to pass through. The interfering sugar which disturbs the sugar measurement is degraded from the blood sample injected through the first injection port 101 by the glucose enzyme injected through the second injection port 102 while passing through the glucosic acid filter unit 103, and the produced glucosic acid is filtered. It is preferred that the glucosic acid which disturbs the glycosylated hemoglobin sensing is maximally removed, and the efficiency of removing the glucosic acid may be controlled by controlling the channel length of the glucosic acid filter unit 103. It is preferred that the glucosic acid filter unit 103 includes a cationic filter that the captures negative glucosic acid to prevent the negative glucosic acid from participating in a reaction. The cationic filter is not particularly limited, but may be used an organic polymer material such as polyester, acryl, and the like, an inorganic material such as carbon fiber, glass fiber, a metal material, and the like or a material such as an organic-inorganic hybrid, and the like.

The sensing chamber unit 104 is a space in which the blood sample which has passed through the glucosic acid filter unit 103 transiently resides. Because the valve unit 107 is present at the end of the sensing chamber unit 104, it is possible to naturally stop the blood sample and accordingly a reaction may occur in the sensing chamber unit 104. The valve unit 107 may include a hydrophobic coating or a hydrophobic patch, but the present invention is not limited thereto. The hydrophobic coating or patch may be selectively provided on the lower substrate 200 or the upper substrate.

The sensing chamber unit 104 includes a space for measuring the concentration of hemoglobin in a blood sample and a space for measuring the concentration of glycosylated hemoglobin. In the space for measuring glycosylated hemoglobin, antibodies which react with glycosylated hemoglobin are immobilized on the upper portion of the sensing electrodes 201, and thus glycosylated hemoglobin in the blood sample reacts with antibodies to be combined with each other and the sensing electrodes 201 may sense the combining to measure the concentration of glycosylated hemoglobin.

The value of glycosylated hemoglobin (that is, ratio of the concentration of glycosylated hemoglobin to the concentration of hemoglobin) may be calculated as the following Mathematical Formula 1.

[Mathematical Formula 1]

% HbA1c=HbA1c/Hb×100(%)

Wherein,

% HbA1c is a value(%) of glycosylated hemoglobin,

HbA1c is the concentration of glycosylated hemoglobin in blood, and

Hb is the concentration of hemoglobin in blood.

The pump unit 108 is connected to the sensing chamber unit 104 to serve to forcing flow the blood sample which has completed the reaction in the sensing chamber unit 104 to the exhaust port 105.

The pump unit 108 may include a washing solution storing unit 108a and a gas generating unit 108b. Water or a buffer solution may be injected through a third injection port 108c into the washing solution storing unit 108a, and a material for generating gas may be injected through a fourth injection port (not shown) into the gas generating unit 108b. The gas generating unit 108b may be disposed so as to correspond to the heater 202.

If electrical signals are applied from an external power source apparatus, and then that the heater 202 generates heat, the pump unit 108 may use gas produced from the material for generating gas to supply the washing solution to the sensing chamber unit 104. For example, carbon dioxide gas may be generated by reactions of paraffin, citric acid, and carbonate to forcingly push out the washing solution to the sensing chamber unit 104. The forced fluid caused by operating the pump unit 108 enables washing of the sensing chamber unit 104, the whole replacement of the blood sample, and the like.

In order to prevent the blood sample which has passed through the glucosic acid filter unit 103 from flowing to the pump unit 108, a fluid separation unit (not shown) may be additionally included between the glucosic acid filter unit 103 and the pump unit 108.

Although not shown, a temperature sensor for controlling the operation of the heater 202 may be disposed around the heater 202, and electrode pads for applying or transferring electrical signals to the heater and the temperature sensor may be further provided.

The exhaust port 105 is for discharging the blood sample which has completed the reaction and/or analysis in the sensing chamber unit 104 from the microfluidic control device to the outside, and may further include a waste liquid chamber 106 which is a space for storing a blood sample discarded before being discharged.

Separate holes which aid the circulation of air in order to help transfer the fluid may be additionally included on the upper substrate 100.

The upper substrate 100 and the lower substrate 200, on which the above-described micro structures are formed, are combined with each other by an adhesive member 300, thereby completing the microfluidic control device according to the present invention. When the upper substrate 100 and the lower substrate 200 are formed of the same material, the adhesive member 300 may be heat, chemicals, an ultrasound wave, and the like. When the substrates 100 and 200 are formed of different materials, the adhesive member 300 may be a liquid-type adhesive material, a powdery adhesive material, and a thin plate-like adhesive material such as paper. When room temperature or low temperature combining is required to prevent modification of biochemical materials (antibodies, and the like) during combining of the upper substrate 100 and the lower substrate 200, a pressure sensitive adhesive carrying out the combining with only pressure may be used.

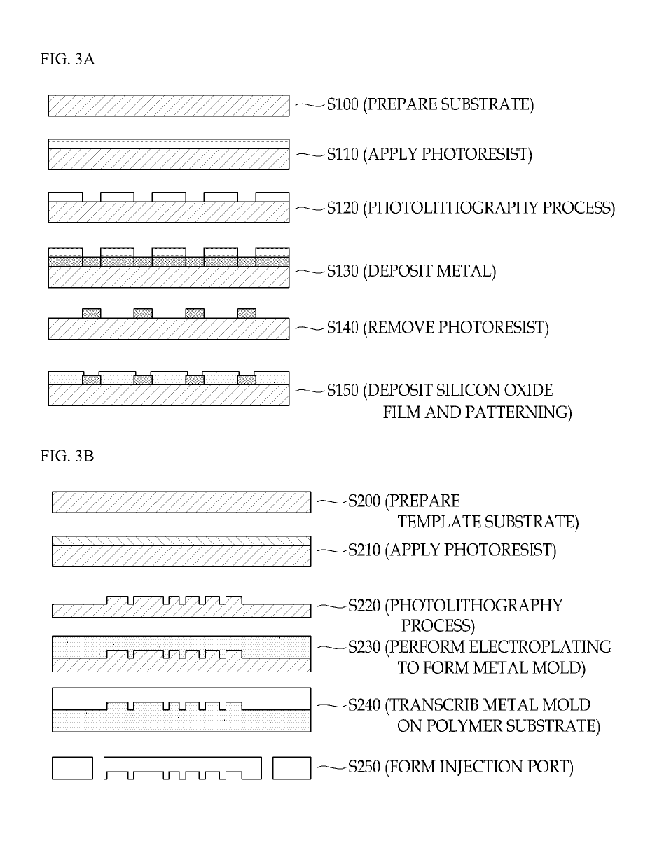

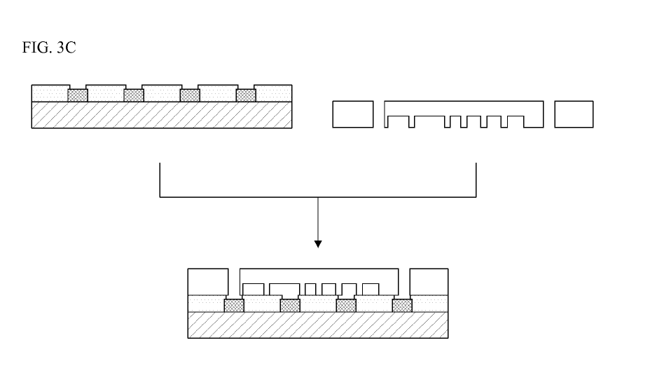

FIGS. 3A to 3C are schematic views schematically illustrating a method for manufacturing a microfluidic control device according to an exemplary embodiment of the present invention.

FIG. 3A shows a process of manufacturing a lower substrate (see 200 of FIG. 1). First, a substrate, for example, a silicon wafer is washed to be prepared (S100) and then a photoresist (PR) is applied thereon (S110). Subsequently, the photolithography process (including masking, exposure, development, and hard baking processes) is used to pattern parts on which a micro electrode array and a micro heater pattern will be formed (S120), an electronic beam or a thermal evaporator is used to deposit metal, for example, Ti/Au metal at a thickness of 0.01 to 1 μm (S130), and then the photoresist is lifted off (S140) to form a desired micro electrode array and a heater pattern. A hydrophobic coating or patch may be formed along with the formation of the micro electrode array and the heater pattern. Subsequently, a silicon oxide film pattern may be additionally formed for surface hydrophobic modification and electrical insulation (S150).

FIG. 3B shows a process of manufacturing an upper substrate (see 100 of FIG. 1). First, a template substrate, for example, a silicon wafer is washed to be prepared (S200) and then a photosensitive photoresist, preferably an epoxy-based photosensitive photoresist is applied thereon (S210). The thickness of the photoresist may be variously controlled (for example: 1 to 1,000 μm) by regulating the viscosity of the photoresist or in proportion to revolutions per minute (for example: 500 to 5,000 rpm) of a spin coating apparatus. The epoxy-based photosensitive photoresist may form a precise pattern without being affected even by additionally performing exposure work after thermal curing occurs and a desired pattern and depth may be readily and rapidly obtained by an exposure process. A representative epoxy-based photoresist may be an SU-8-based negative photoresist. A precise pattern shape (resolution limit of 1 μm or more) may be controlled by a pattern of an exposure mask. An upper substrate mold prototype including a multi-step structure corresponding to a fluid channel shape having an ultraprecise channel depth and pattern shape may be completed by this process (S220).

A metal mold may be manufactured by applying the electroplating process to the multi-step structure of the upper substrate mold prototype manufactured (S230). The electroplating may be performed after forming a seed layer formed of a metal such as titanium (Ti), chromium (Cr), aluminum (Al), gold (Au), and the like. A thickness of the metal mold is sufficient as long as there is no bending or breaking when the metal mold is transcribed on a substrate (for example: polymer substrate) to be transcribed. Subsequently, the template substrate (for example: silicon substrate) is removed by a method such as wet etching, and the like such that only the metal mold is left behind.

The metal mold is transcribed on the substrate (for example: polymer substrate) by a method such as injection molding, hot embossing, casting, and the like (S240). Subsequently, fluid injection holes (see 101 and 102 of FIG. 1), a fluid exhaust hole (105 of FIG. 1), air holes, and the like are formed.

FIG. 3C shows a process of combining the upper substrate manufactured in FIG. 3B with the lower substrate manufactured in FIG. 3A. As show in FIG. 3C, the sensing chamber unit is combined with the electrode array to correspond to each other and the pump unit is combined with the heater pattern to correspond to each other. When the upper and lower substrates are formed of the same material, the substrates may be combined by heat, chemicals, an ultrasound wave, and the like. When the upper and lower substrates are formed of different materials, a liquid-type adhesive material, a powdery adhesive material, and a thin plate-like adhesive material such as paper may be used. When room temperature or low temperature combining is required to prevent modification of biochemical materials (such as antibodies, and the like) during combining of the upper substrate and the lower substrate, a pressure sensitive adhesive carrying out the combining with only pressure may be used.

The method for operating the microfluidic control device of the present invention is carried out as follows.

A blood sample is injected through the first injection port 101 and glucose enzyme is injected through the second injection port 102. The injected blood sample and glucose enzyme are mixed and while passing through the glucosic acid filter unit 103 by natural flow caused by capillary force, the interfering sugar is degraded by the glucose enzyme and the produced glucosic acid is filtered. If the sample which has passed through the glucosic acid filter unit 103 is transferred to the sensing chamber unit 104 and the flow of the sample is stopped by the valve unit 107, the concentration of hemoglobin and glycosylated hemoglobin may be measured by the sensing electrode 106 provided in the sensing chamber unit 104. Antibodies are provided on the sensing electrode of the chamber unit for measuring glycosylated hemoglobin, and thus the concentration of glycosylated hemoglobin may be measured by reaction of glycosylated hemoglobin with antibodies. The blood sample which has sufficiently reacted in the sensing chamber unit 104 is discarded into the waste liquid chamber 106 by operating the pump unit 108. The discard of the blood sample may be carried out by forced fluid of washing solution caused by pressure of gas which has been generated by operating the pump unit 108.

From the foregoing, it will be appreciated that various embodiments of the present invention have been described herein for purposes of illustration, and that various modifications may be made without departing from the scope and spirit of the presentinvention. Accordingly, the various embodiments disclosed herein are not intended to be limiting, with the true scope and spirit being indicated by the following claims.

What is claimed is:

1. A microfluidic control device for measuring glycosylated hemoglobin, comprising:
a first substrate; and
a second substrate provided on the first substrate and having a first injection port into which a blood sample is injected, a second injection port into which glucose enzyme is injected, a glucosic acid filter unit which is connected to the first injection port and the second injection port and captures glucosic acid which has been produced by reaction of the blood sample with the glucose enzyme, a sensing chamber unit which is connected to the glucosic acid filter unit and electrochemically measures the concentration of hemoglobin and glycosylated hemoglobin in the blood sample introduced from the glucosic acid filter unit, an exhaust port which is connected to the sensing chamber unit and discharges the blood sample which has completed the reaction in the sensing chamber unit, a valve unit for controlling the flow of fluid between the sensing chamber unit and the exhaust port, and a pump unit for forcingly flowing the blood sample which has completed the reaction in the sensing chamber unit to the exhaust port.

2. The microfluidic control device of claim 1, wherein the glucosic acid filter unit comprises a cationic filter for capturing glucosic acid.

3. The microfluidic control device of claim 1, wherein the first substrate comprises a sensing electrode for sensing electrochemical signals, and the sensing electrode corresponds to the sensing chamber unit.

4. The microfluidic control device of claim 1, wherein the sensing chamber unit for measuring glycosylated hemoglobin comprises antibodies which react with glycosylated hemoglobin.

5. The microfluidic control device of claim 1, which further comprises a filter for capturing red blood cells from the blood sample and separating giant proteins before the blood sample is injected into the first injection port.

6. The microfluidic control device of claim 1, wherein the first substrate comprises a heater, the pump unit comprises a washing solution storing unit and a gas generating unit, water or a buffer solution is injected into the washing solution storing unit, and the gas generating unit comprises a gas generating material and corresponds to the heater.

7. The microfluidic control device of claim 1, wherein the first substrate comprises a hydrophobic coating or a hydrophobic patch, and the hydrophobic coating or the hydrophobic patch corresponds to the valve unit.

8. A method for manufacturing the microfluidic control device for measuring glycosylated hemoglobin of claim 1, comprising:
preparing a first substrate;
preparing a second substrate, comprising applying a photosensitive photoresist on a template substrate, forming a channel structure corresponding to a glucosic acid filter unit, a sensing chamber unit, a valve unit, and a pump unit on the template substrate by a photolithography process, performing electroplating on the channel structure to form a metal mold and removing the template substrate, transcribing the metal mold on a second precursor substrate, and forming a first injection port, a second injection port, and an exhaust port on the second precursor substrate; and
combining the second substrate on the first substrate to form the glucosic acid filter unit, the sensing chamber unit, the valve unit, and the pump unit.

9. The method of claim 8, wherein the preparing of the first substrate comprises applying a photoresist on a first precursor substrate, patterning the photoresist by a photolithography process, and forming a metal pattern for sensing electrodes, and the combining of the second substrate on the first substrate is performed such that the channel structure corresponding to the sensing chamber unit corresponds to the metal pattern for sensing electrodes.

10. The method of claim 8, wherein the preparing of the first substrate comprises applying a photoresist on a first precursor substrate, patterning the photoresist by a photolithography process, and forming a metal pattern for a heater,
the forming of the channel structure corresponding to the pump unit on the second substrate comprises forming a channel structure corresponding to a washing solution storing unit and a gas generating unit, and
the combining of the second substrate on the first substrate is performed such that the channel structure corresponding to the gas generating unit corresponds to the metal pattern for a heater.

11. The method of claim 8, wherein the preparing of the first substrate comprises forming a hydrophobic coating or a hydrophobic patch, and
the combining of the second substrate on the first substrate is performed such that the channel structure corresponding to the valve unit corresponds to the hydrophobic coating or the hydrophobic patch.

12. The method of claim 8, wherein the template substrate is a silicon substrate and the second precursor substrate is a polymer substrate.

13. The method of claim 9, wherein the first precursor substrate is a silicon substrate.

14. The method of claim 10, wherein the first precursor substrate is a silicon substrate.

15. A method for operating a microfluidic control device for measuring glycosylated hemoglobin, comprising:
injecting a blood sample into a first injection port and injecting glucose enzyme into a second injection port to naturally flow the blood sample and the glucose enzyme to a glucosic acid filter unit by capillary force, by using the microfluidic control device for measuring glycosylated hemoglobin of claim 1;
capturing the glucosic acid produced while passing through the glucosic acid filter unit;
transferring the blood sample which has passed through the glucosic acid filter unit to the sensing chamber unit by capillary force and electrochemically measuring the hemoglobin concentration and the glycosylated hemoglobin concentration; and
forcingly flowing the blood sample which has been reacted in the sensing chamber unit to the exhaust port by operating the pump unit.

16. The method of claim 15, wherein the forcingly flowing of the blood sample which has been reacted in the sensing chamber unit to the exhaust port is performed by forced fluid of the washing solution injected into the pump unit caused by pressure of gas which has been generated by operating the pump unit.
